(19) United States
(12) Patent Application Publication
(10) Pub. No.: US 2024/0148825 A1
(43) Pub. Date: May 9, 2024

(54) METHODS AND COMPOSITIONS FOR TREATMENT OF AUTOIMMUNE CONDITIONS

(71) Applicant: UNIVERSITÄTSMEDIZIN DER JOHANNES GUTENBERG-UNIVERSITÄT MAINZ, Mainz (DE)

(72) Inventors: Wolfram RUF, Mainz (DE); Nadine MÜLLER-CALLEJA, Mainz (DE); Karl LACKNER, Mainz (DE); Julia WEINMANN-MENKE, Mainz (DE)

(73) Assignee: UNIVERSITÄTSMEDIZIN DER JOHANNES GUTENBERG-UNIVERSITÄT MAINZ, Mainz (DE)

(21) Appl. No.: 18/567,625

(22) PCT Filed: Jun. 9, 2022
(86) PCT No.: PCT/IB2022/055391

§ 371 Date: Dec. 6, 2023

Related U.S. Application Data
(60) Provisional application No. 63/208,929, filed on Jun. 9, 2021.

(51) Int. Cl. A61K 38/17; A61K 45/06; A61P 37/06

(52) U.S. Cl. CPC A61K 38/1767; A61K 45/06; A61P 37/06

(57) ABSTRACT

The current disclosure provides methods and composition for treatment of autoimmune and inflammatory conditions, including systemic lupus erythematosus and antiphospholipid syndrome. Certain aspects of the disclosure are directed to methods for treatment of an autoimmune or inflammatory condition comprising administering a composition comprising a therapeutically effective amount of NAPc2 or NAPc2/proline. Further aspects include pharmaceutical compositions comprising NAPc2 or NAPc2/proline and, in some cases, one or more additional anti-inflammatory agents.

### CROSS-REFERENCE TO RELATED APPLICATIONS

This application claims priority to and the benefit of U.S. Provisional Application No. 63/208,929, filed Jun. 9, 2021, the contents of which is incorporated into the present application in its entirety.

### SEQUENCE LISTING

The instant application contains a Sequence Listing which has been submitted in ASCII format and is hereby incorporated by reference in its entirety. Said ASCII copy, created on Jun. 8, 2021, is named ARCAP0067WO_Sequence_Listing.txt and is 1,943 bytes in size.

### BACKGROUND

### I. Field of the Invention

Aspects of this invention relate to at least the fields of immunology, hematology, and medicine.

### II. Background

Lipid-reactive antibodies transiently appear in infectious diseases, but clonal evolution of persistent autoimmune antiphospholipid antibodies (aPL) causes antiphospholipid syndrome (APS) characterized by severe thrombo-embolic and microangiopathic complications, pregnancy morbidity, and fetal loss. Systemic lupus erythematosus (SLE) is a chronic autoimmune disease and is characterized by the presence of self-reactive autoantibodies, including antiphospholipid antibodies and anti-dsDNA antibodies.

Recognized herein is a need for methods and compositions for treatment of subjects having an autoimmune or inflammatory condition, including systemic lupus erythematosus and antiphospholipid syndrome.

### SUMMARY

The current disclosure fulfils certain needs by providing methods and compositions for treating or preventing autoimmune or inflammatory conditions. Accordingly, aspects of the disclosure provide methods and compositions for treating a subject for an autoimmune or inflammatory condition, for example systemic lupus erythematosus or antiphospholipid syndrome. In certain aspects, disclosed are compositions comprising NAPc2 or NAPc2/proline and methods for use of such compositions in the treatment of autoimmune and inflammatory conditions including systemic lupus erythematosus and antiphospholipid syndrome.

Embodiments of the present disclosure include methods for treating a subject having an autoimmune or inflammatory condition, methods for treating a subject for systemic lupus erythematosus, methods for treating a subject for antiphospholipid syndrome, methods for evaluating an efficacy of an anti-inflammatory treatment, pharmaceutical compositions, polynucleotides, and nucleic acids. Methods of the disclosure can include at least 1, 2, 3, or more of the following steps: diagnosing a subject for an autoimmune or inflammatory condition, measuring one or more symptoms of an autoimmune or inflammatory condition in a subject, detecting antiphospholipid antibodies in a biological sample from a subject, detecting anti-cardiolipin antibodies in a biological sample from a subject, administering NAPc2 to a subject, administering a NAPc2 variant to a subject, administering rNAPc2 to a subject, administering an anti-inflammatory agent to a subject, administering an anticoagulant to a subject, and administering a coagulation factor to a subject. It is specifically contemplated that one or more of the preceding steps may be omitted in certain embodiments.

Disclosed herein, in some embodiments, is a method for treating a subject for an autoimmune or inflammatory condition, the method comprising administering to the subject a therapeutically effective amount of a pharmaceutical composition comprising nematode anticoagulant protein c2 (NAPc2) or NAPc2/proline. In some embodiments, the pharmaceutical composition comprises NAPc2. In some embodiments, the pharmaceutical composition comprises NAPc2/proline. The pharmaceutical composition may comprise one or more additional therapeutics. In some embodiments, the method further comprises administering an additional anti-inflammatory agent to the subject. In some embodiments, the additional anti-inflammatory agent is a non-steroidal anti-inflammatory drug (NSAID). In some embodiments, the pharmaceutical composition does not comprise any additional therapeutics. The pharmaceutical composition may comprise one or more pharmaceutically acceptable excipients.

In some embodiments, the subject was diagnosed with an autoimmune or inflammatory condition. The subject may be or have been diagnosed with the autoimmune or inflammatory condition by any means known in the art. In some embodiments, the subject was determined to have one or more symptoms of an autoimmune or inflammatory condition. A symptom of an autoimmune or inflammatory condition may be, for example, fatigue, skin lesions, rash, fever, thrombosis (e.g., venous thrombosis, pulmonary embolism), thrombocytopenia, high blood pressure, kidney failure, or recurrent miscarriage. One or more of these symptoms may be excluded from embodiments of the disclosure. In some embodiments, the pharmaceutical composition is administered to the subject following the onset of the symptoms. In some embodiments, the subject was not diagnosed with an autoimmune or inflammatory condition. In some embodiments, the pharmaceutical composition is administered prior to the onset of any symptoms of an autoimmune or inflammatory condition. For example, the pharmaceutical composition may be administered to subject at risk for having or developing an autoimmune or inflammatory condition. In some embodiments, the subject was determined to have antiphospholipid antibodies. In some embodiments, the method further comprises detecting the presence of antiphospholipid antibodies in the subject.

In some embodiments, the subject was previously treated for an autoimmune or inflammatory condition with a previous treatment (e.g., a previous anti-inflammatory agent). In some embodiments, the subject was determined to be resistant to the previous treatment. In some embodiments, the subject was not previously treated for an autoimmune or inflammatory condition. In some embodiments, the subject is treated with a pharmaceutical composition comprising NAPc2 or NAPc2/proline together with 1, 2, 3, 4, 5, 6, 7, or more additional therapeutics (e.g., anti-inflammatory agents, anticoagulants, etc.).

In some embodiments, the pharmaceutical composition is administered via subcutaneous injection. In some embodiments, the pharmaceutical composition is administered via intravenous infusion. In some embodiments, the pharmaceutical composition is administered to the subject every 1, 2, 3, 4, 5, 6, 7, 8, 9, 10, 12, 13, or 14 days. In some embodiments, the pharmaceutical composition is administered to the subject every other day. In some embodiments, the pharmaceutical composition is administered on a first, second, third, fourth, fifth, sixth, seventh, eighth, ninth, tenth, eleventh, twelfth, thirteenth, and/or fourteenth day. In some embodiments, the pharmaceutical composition is administered on a first day, a third day, and a fifth day.

In some embodiments, the NAPc2 or NAPc2/proline is administered to the subject at a dose of at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or 15.0 μg/kg. In some embodiments, the NAPc2 or NAPc2/proline is administered at a dose of between 5 μg/kg and 10 μg/kg. In some embodiments, the NAPc2 or NAPc2/proline is administered at a dose of about 10 μg/kg. In some embodiments, the NAPc2 or NAPc2/proline is administered at a dose of about 7.5 μg/kg. In some embodiments, the NAPc2 or NAPc2/proline is administered at a dose of about 5 μg/kg. In some embodiments, the NAPc2 or NAPc2/proline is administered on a first day, a third day, and a fifth day. In some embodiments, the NAPc2 or NAPc2/proline is administered at a dose of about 7.5 μg/kg on a first day, 5 μg/kg on a third day, and 5 μg/kg on a fifth day.

In some embodiments, the method further comprises administering an additional anticoagulant to the subject. In some embodiments, the additional anticoagulant is a VKORC1 inhibitor, a thrombin inhibitor, or a factor Xa inhibitor. In some embodiments, the additional anticoagulant is warfarin, heparin or synthetic analogs thereof, rivaroxaban, dabigatran, apixaban, or edoxaban. The method may comprise administering 1, 2, 3, 4, 5, or more additional anticoagulants.

In some embodiments, the method does not comprise administering an additional anticoagulant to the subject. For example, in some embodiments, the method does not comprise administering a VKORC1 inhibitor, a thrombin inhibitor, or a factor Xa inhibitor. In some embodiments, the method does not comprise administering a warfarin, heparin or synthetic analogs thereof, rivaroxaban, dabigatran, apixaban, or edoxaban.

Also disclosed herein, in some embodiments, is a method for treating a subject for antiphospholipid syndrome, the method comprising administering to the subject a therapeutically effective amount of a pharmaceutical composition comprising nematode anticoagulant protein c2 (NAPc2) or NAPc2/proline. Further disclosed, in some embodiments, is a method for treating a subject for systemic lupus erythematosus, the method comprising administering to the subject a therapeutically effective amount of a pharmaceutical composition comprising nematode anticoagulant protein c2 (NAPc2) or NAPc2/proline. In some embodiments, the pharmaceutical composition comprises NAPc2. In some embodiments, the pharmaceutical composition comprises NAPc2/proline. In some embodiments, the method further comprises, prior to administering the pharmaceutical composition, detecting the presence of antiphospholipid antibodies in a biological sample from the subject. The antiphospholipid antibodies may be detected in the biological sample via any method known in the art. In some embodiments, detecting the antiphospholipid antibodies comprises an enzyme linked immunosorbent assay (ELISA).

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows results from next generation sequencing analysis of aPL HL5B-induced activation of monocytic MM1 cells and inhibition by rNAPc2 and anti-TF antibody 10H10.

FIG. 2 shows the effect of rNAPc2 treatment of MRL/lpr mice on aPL titers. Mice were treated with 0.5 mg/kg every second day starting at an age of 11 weeks; ***P<0.001; ****P<0.0001.

FIGS. 3A and 3B show the effect of rNAPc2 treatment of MRL/lpr mice on anti-cardiolipin (FIG. 3A) and β2GPI (FIG. 3B) titers. Mice were treated with 0.5 mg/kg every second day starting at an age of 11 weeks; **P<0.01; ****P<0.0001.

FIGS. 4A and 4B. FIG. 4A shows the effect of rNAPc2 treatment on circulating B cells reactive with fluorescently labeled phospholipid in MRL/lpr mice. Cells were stained in the presence of unlabeled competitor sEPCR/PC, sEPCR/LBPA, or β2GPI. FIG. 4B shows results from staining of B cells with fluorescently labeled β2GPI with or without sEPCR-PC or sEPCR-LBPA.

FIG. 5 shows results from analysis of albuminuria in MRL/lpr mice sham or rNAPc2 treated started at 11 weeks of age.

FIG. 6 shows evaluation of glomerular and tubular histology by a semiquantitative scoring, as described [1]. Pooled data are shown from two cohorts treated with rNAPc2 versus saline; ****P<0.0001.

FIG. 7 shows evaluation of kidney immune cell infiltration scored on sections stained for macrophages, T cells, and B cells, as described [1]. Pooled data are shown from the two cohorts treated with rNAPc2 versus saline; *P<0.05, **P<0.01.

FIGS. 8A-8D show the effect of NAPc2 versus heparin therapy on the development of aPL in lupus-prone mice. FIGS. 8A and 8B show antibody reactivity with cardiolipin or β2GPI of serum samples obtained from the mice treated as indicated. FIGS. 8C and 8D show staining of peripheral blood B cells for phospholipid or β2GPI reactivity in the absence or presence of excess β2GPI, sEPCR, or sEPCR-LBPA. The proportion of CD19 positive cells are shown.

FIGS. 9A-9C show results from next generation sequencing analysis of monocytic MM1 cells stimulated by aPL HL5B or HL7G, which is cross-reactive with β2GPI, in plasma, with and without NAPc2 inhibition. FIG. 9A shows a heatmap of differently regulated transcripts induced by HL5 stimulation. FIG. 9B shows transcripts still induced in aPL HL5B stimulated cells in the presence of NAPc2. FIG. 9C shows transcripts still induced in aPL HL7G stimulated cells in the presence of NAPc2.

### DETAILED DESCRIPTION

The present disclosure is based at least in part on the discovery that NAPc2 abolishes the proinflammatory activation of monocytes by antiphospholipids (aPL), suppresses the expansion of cardiolipin-reactive and β2 glycoprotein I-reactive auto-antibodies in a mouse model of systemic lupus erythematosus (SLE), and prevents kidney disease in a preclinical model of SLE. Thus, as disclosed herein, NAPc2 not only acts as inhibitor of TF-dependent thrombosis, but also prevents the development of pathogenic auto-antibodies and organ pathologies in autoimmune disease. Accordingly, aspects of the present disclosure are directed to methods for treating a subject for an autoimmune or inflammatory condition, including systemic lupus erythematosus and antiphospholipid syndrome, comprising administering NAPc2 or a variant thereof (e.g., NAPc2/proline) to the subject.

### I. Proteins

As used herein, a “protein” or “polypeptide” refers to a molecule comprising at least four amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide. The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more contiguous amino acids or nucleic acids, or any range derivable therein, of SEQ ID NO:1 and/or SEQ ID NO:2. Polynucleotides of the disclosure may encode a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or being at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more contiguous amino acids or nucleic acids, or any range derivable therein, of SEQ ID NO:1 and/or SEQ ID NO:2

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) of SEQ ID NO:1 and/or SEQ ID NO:2.

In some embodiments, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids of SEQ ID NO:1 and/or SEQ ID NO:2.

In some embodiments, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids of SEQ ID NO:1 and/or SEQ ID NO:2 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NO:1 and/or SEQ ID NO:2.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or 83 of any of SEQ ID NO:2 and/or SEQ ID NO:3 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids or nucleotides of any of SEQ ID NO:1 and/or SEQ ID NO:2.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated herein that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

B. Nematode-Extracted Anticoagulant Proteins and NAPc2

Aspects of the present disclosure are directed to compositions comprising one or more Nematode-extracted Anticoagulant Proteins (NAPs) and methods of use thereof. In some embodiments, disclosed are methods for treatment comprising administering a subject with a pharmaceutical composition comprising one or more NAPs. In some embodiments, NAPs of the present disclosure are one or more of those described in U.S. Pat. No. 5,866,542, incorporated herein by reference in its entirety. In some embodiments, the disclosed methods and compositions comprise NAPc2. In some embodiments, the disclosed methods and compositions comprise NAPc2/proline.

As used herein, NAPc2, (SEQ ID NO:1) describes a single-chain, non-glycosylated 85 amino acid protein (MW=9732 Da). “rNAPc2” describes a recombinant NAPc2 protein. Without wishing to be bound by theory, rNAPc2 is understood to inhibit the activity of the TF:Factor (F) VIIa complex that initiates the TF pathway in coagulation, and other key pathways, through the formation of a quaternary complex following binding to zymogen FX. Also disclosed herein are variants of rNAPc2. In some embodiments, the disclosed therapeutic compositions comprise a protein having at least or at most 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity (or any range or value derivable therein) to NAPc2 (SEQ ID NO:1) or NAPc2/proline (SEQ ID NO:2). In some embodiments, disclosed are compositions comprising NAPc2/proline. “NAPc2/proline” (SEQ ID NO:2) refers to a variant of NAPc2, which has been modified to add a proline residue to the C-terminus of the sequence of NAPc2.

TABLE 1
NAPc2 and NAPc2 variant sequences

| | SEQ ID | |
|---|---|---|
| Protein | NO | Sequence |
| NAPc2 | 1 | KATMQCGENEKYDSCGSKECDKKCKYDGVEEEDDEEPNVPCL |
| | | VRVCHQDCVCEEGFYRNKDDKCVSAEDCELDNMDFIYPGTRN |
| NAPc2/ | 2 | KATMQCGENEKYDSCGSKECDKKCKYDGVEEEDDEEPNVPCL |
| proline | | VRVCHQDCVCEEGFYRNKDDKCVSAEDCELDNMDFIYPGTRNP |

### II. Autoimmune and Inflammatory Conditions

Aspects of the present disclosure are directed to methods for treatment of an autoimmune or inflammatory condition. As used herein, the terms “autoimmune disease,” “autoimmune condition,” and “inflammatory condition” are used interchangeably. In some embodiments, disclosed are methods for treatment of an autoimmune or inflammatory condition comprising administering NAPc2 or NAPc2/proline to a subject.

The autoimmune condition or inflammatory condition amenable for treatment may include, but not be limited to, conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes.

Additional autoimmune and inflammatory conditions contemplated herein include sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, antiphospholipid syndrome (also “anti-phospholipid antibody syndrome”), allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, cytokine storm, contact hypersensitivity, asthmatic airway hyperreaction, and endometriosis.

In some embodiments, the autoimmune or inflammatory condition is systemic lupus erythematosus. In some embodiments, the autoimmune or inflammatory condition is antiphospholipid syndrome (APS; also “the antiphospholipid syndrome”).

### III. Treatment of Autoimmune and Inflammatory Conditions

Aspects of the present disclosure are directed to methods for treatment of a subject having an autoimmune or inflammatory condition, including any autoimmune or inflammatory condition disclosed herein, for example systemic lupus erythematosus or antiphospholipid syndrome. In some embodiments, disclosed are methods for treatment of a subject having an autoimmune or inflammatory condition comprising providing a therapeutically effective amount of NAPc2 or a variant thereof (e.g., NAPc2/proline).

Certain embodiments of the disclosure are directed to treatment of subjects having one or more symptoms of an autoimmune or inflammatory condition. In embodiments where the autoimmune or inflammatory condition is systemic lupus erythematosus, the symptoms may include, but are not limited to, fatigue, skin lesions, rash, kidney failure, and fever. In embodiments where the autoimmune or inflammatory condition is antiphospholipid syndrome, the symptoms may include, but are not limited to, thrombosis (e.g., venous thrombosis, pulmonary embolism), thrombocytopenia, high blood pressure, kidney failure, and recurrent miscarriage. In some embodiments, a subject has been diagnosed with an autoimmune or inflammatory condition. In some embodiments, a subject has not been diagnosed with an autoimmune or inflammatory condition. In some embodiments, a subject is at risk for having or developing an autoimmune or inflammatory condition.

In some embodiments, the subject was previously treated for an autoimmune or inflammatory condition. In some embodiments, a composition comprising NAPc2 or NAPc2/proline is provided to a subject having an autoimmune or inflammatory condition, where the subject previously suffered from and was treated for the autoimmune or inflammatory condition with one or more anti-inflammatory agents. In some embodiments, the one or more anti-inflammatory agents did not comprise NAPc2 or NAPc2/proline. In some embodiments, the subject was determined to be resistant to the previously provided one or more anti-inflammatory agents.

In some embodiments, a subject treated for an autoimmune or inflammatory condition is at least, is at most, or is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 years of age, or any range derivable therein.

In some embodiments, a subject is administered a pharmaceutical composition comprising NAPc2 or a variant thereof (e.g., NAPc2/proline). The pharmaceutical composition may be administered in a therapeutically effective amount. In some embodiments, the NAPc2 or NAPc2/proline is provided at a dose of at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or 15.0 μg/kg or mg/kg, or any range or value derivable therein. The pharmaceutical composition may be administered to a subject every day, every other day, every third day, or every fourth day. In some embodiments, the pharmaceutical composition is administered to the subject on a first day, a third day, and a fifth day. The NAPc2 or variant thereof (e.g., NAPc2/proline) may be administered at the same dose on each day or at different doses. In some embodiments, the NAPc2 or variant thereof (e.g., NAPc2/proline) is provided at a first dose on a first day and a second dose on each subsequent day of treatment. In some embodiments, the NAPc2 or variant thereof (e.g., NAPc2/proline) is provided at a first dose on a first day and a second dose on a third day and a fifth day. In some embodiments, the NAPc2 or variant thereof (e.g., NAPc2/proline) is provided at a dose of about 7.5 μg/kg on a first day, about 5.0 μg/kg on a third day, and about 5.0 μg/kg on a fifth day.

Aspects of the disclosure are directed to administration of one or more anti-inflammatory agents. In some embodiments, an anti-inflammatory agent of the disclosure is NAPc2 or a variant thereof (e.g., NAPc2/proline). In some embodiments, the anti-inflammatory agent is NAPc2. In some embodiments, the anti-inflammatory agent is NAPc2/proline. Additional anti-inflammatory agents are known in the art and contemplated herein, examples of which include corticosteroids, non-steroidal anti-inflammatory drugs (NSAIDs), TNFα inhibitors (e.g., Adalimumab, Certolizumab, Etanercept, Golimumab, Infliximab), and other biologic anti-inflammatory agents (e.g., belimumab).

### IV. Administration of Therapeutic Compositions

The therapy provided herein may comprise administration of a single therapeutic agent (e.g., NAPc2, NAPc2/proline) or a combination of therapeutic agents, such as NAPc2 (or NAPc2/proline) and an additional anti-inflammatory agent. The therapies may be administered in any suitable manner known in the art. For example, each of a first and second therapy may be administered sequentially (at different times) or concurrently (at the same time). In some embodiments, the first and second therapies are administered in a separate composition. In some embodiments, the first and second therapies are in the same composition.

Embodiments of the disclosure relate to compositions and methods comprising therapeutic compositions. A therapeutic composition may comprise a single therapeutic agent (e.g., NAPc2, NAPc2/proline) or multiple different therapeutic agents. The different agents may be administered in one composition or in more than one composition, such as 2 compositions, 3 compositions, or 4 compositions. Various combinations of the agents may be employed.

The therapeutic agents of the disclosure (e.g., NAPc2, NAPc2/proline) may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the therapeutic agent (e.g., NAPc2, NAPc2/proline) is administered subcutaneously. In some embodiments, the therapeutic agent (e.g., NAPc2, NAPc2/proline) is administered intravenously. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 1 μg/kg to 200 μg/kg can affect the protective capability of these agents. It is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. In some embodiments, an effective dose is at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 μg/kg. Furthermore, such doses can be administered at multiple times during a day, and/or on multiple days, weeks, or months.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### V. General Pharmaceutical Compositions

In some embodiments, pharmaceutical compositions are administered to a subject. Different aspects may involve administering an effective amount of a composition to a subject. In some embodiments, NAPc2 (or NAPc2/proline) may be administered to the subject to protect against or treat a condition (e.g., an autoimmune or inflammatory condition). Such compositions may be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, or in addition, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate certain embodiments of the invention. It should be appreciated by those of skill in the art that the techniques disclosed in the examples which follow represent techniques discovered to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—rNAPc2 Effects on aPL Induced Gene Expression in Monocytes

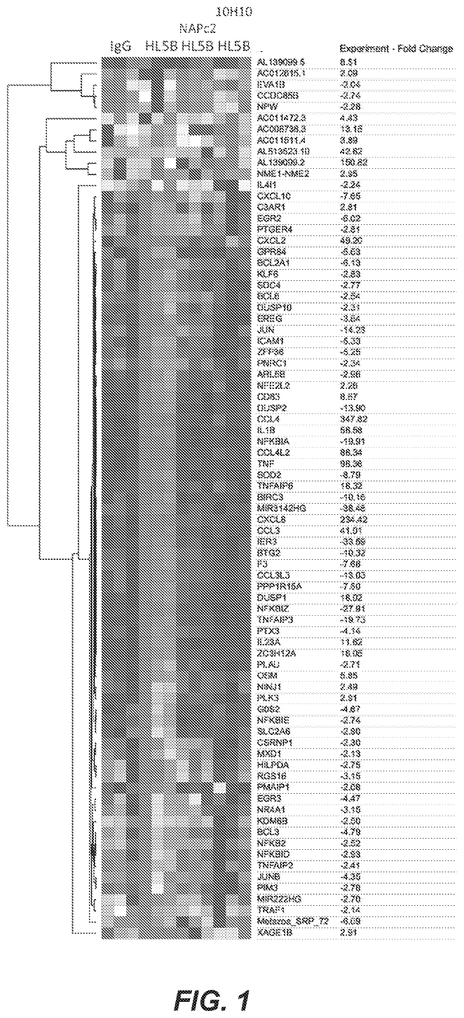

The effects of recombinant NAPc2/proline (rNAPc2) on pro-inflammatory activation of monocytic MM1 cells by antiphospholipid antibodies (aPL) were evaluated using an established assay procedure in vitro [1]. MM1 cells were suspended in medium containing human plasma to provide a source of FX recognized by rNAPc2 for inhibition of TF-FVIIa. Cells were then stimulated for 1 hour with aPL HL5B or IgG control and processed for genome wide transcript profiling by next generation sequencing (NGS). In separate reactions, the inhibitory effects of rNAPc2 and of anti-TF 10H10, an established inhibitor of aPL induced TF activation and endosomal signaling [2, 3], were evaluated. The induction of procoagulant (TF, F3) and proinflammatory responses was blocked by rNAPc2 as efficiently as by the TF antibody 10H10 (FIG. 1). Thus, NAPc2 is an effective inhibitor of aPL induced signaling in monocytic cells.

### Example 2—rNAPc2 Inhibition of Auto-Antibody Production and Autoimmune Pathology in the MRL/lpr Mouse Model of SLE

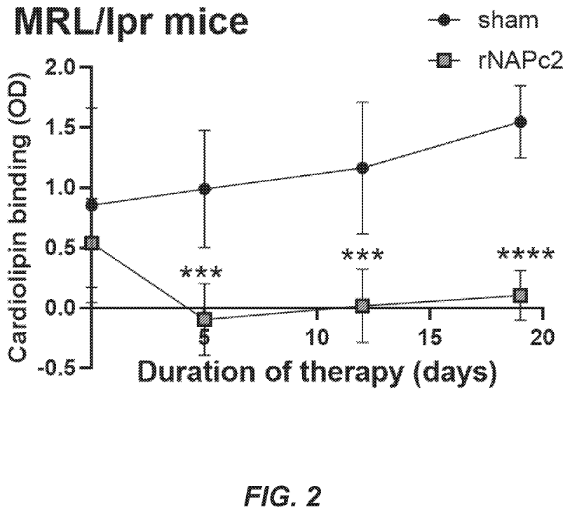

The inhibition of lupus pathology was evaluated in a mouse model of genetic susceptibility for autoimmune disease. MRL/lpr mice develop a SLE-like syndrome with high titer aPL and β2 glycoprotein I (β2GPI) autoantibodies [1]. MRL/lpr mice develop aPL at an age of 5-6 weeks and aPL titers persist during the development of the severe SLE-like syndrome that requires euthanasia of mice before they reach an age of 15-16 weeks. MRL/lpr mice were treated at an age of 11 weeks with 0.5 mg/kg rNAPc2 or saline control every other day for 3 weeks. At the time of randomization to therapy, both groups had similar anti-cardiolipin aPL titers, but aPL titers rapidly declined in rNAPc2-treated mice within one week of therapy and did not reappear for the duration of the experiment (FIG. 2).

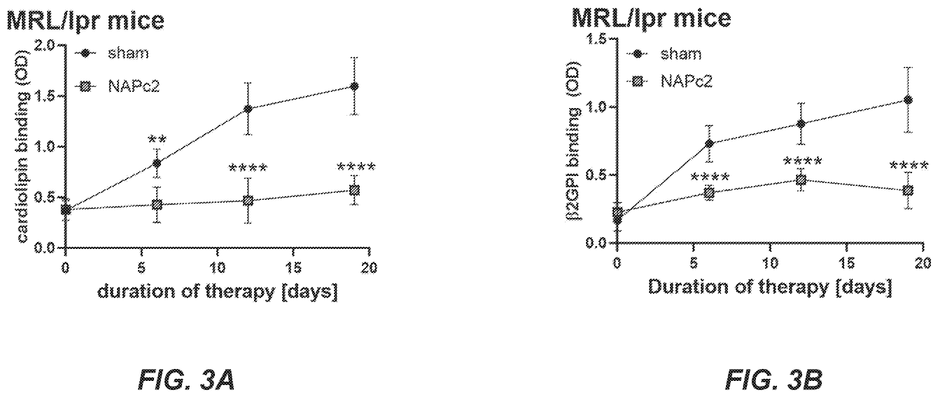

In a second cohort, MRL/lpr mice were similarly randomized for treatment and the reactivity of serum samples against cardiolipin or β2GPI was determined during therapy with rNAPc2. Whereas antibody titers markedly increased over time in sham treated mice, mice randomized to receive rNAPc2 therapy continued to produce very low titers of both cardiolipin (FIG. 3A) and β2GPI (FIG. 3B) auto-antibodies.

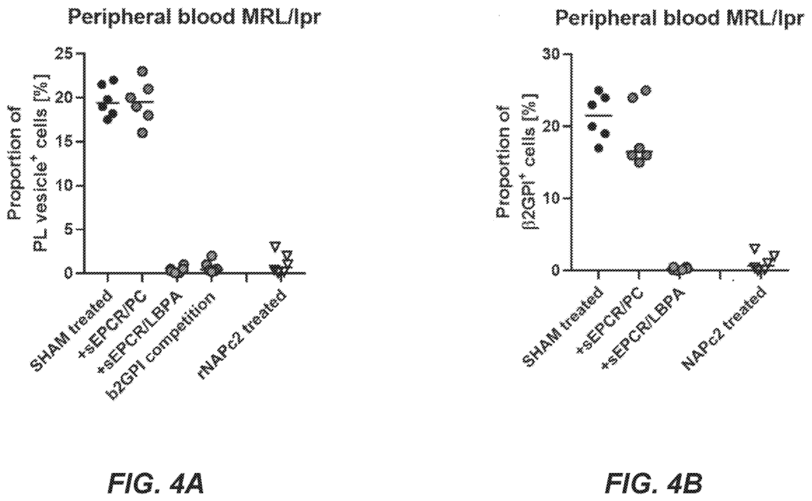

Autoimmune aPL are produced by B1 cells that circulate in the blood [1]. At the end of the above-described experiment, the effect of rNAPc2 therapy on the reactivity of circulating B1 cells was measured with either fluorescently labelled phospholipid vesicles or β2GPI. As expected from the measured aPL titers in these mice, sham treated MRL/lpr mice showed a high percentage of phospholipid and β2GPI reactive circulating B cells (FIGS. 4A and 4B). Staining of B cells by phospholipid vesicles was not blocked by soluble EPCR loaded with phosphatidylcholine (sEPCR-PC), but prevented by either the pathogenic target of aPL, LBPA-loaded sEPCR, or unlabeled β2GPI (FIG. 4A). In line with measured antibody titers, no phospholipid reactive B cells were detected in rNAPc2 treated MRL/lpr mice. Similarly, β2GPI reactive B cells were only found in sham treated, but not rNAPc2 treated MRL/lpr mice with lupus like pathology (FIG. 4B). β2GPI staining was specifically prevented by sEPCR-LBPA, indicating that aPL in this mouse model of autoimmunity have dual reactivity with EPCR-LBPA and β2GPI and that rNAPc2 treatment effectively prevents the development of autoimmune antibodies.

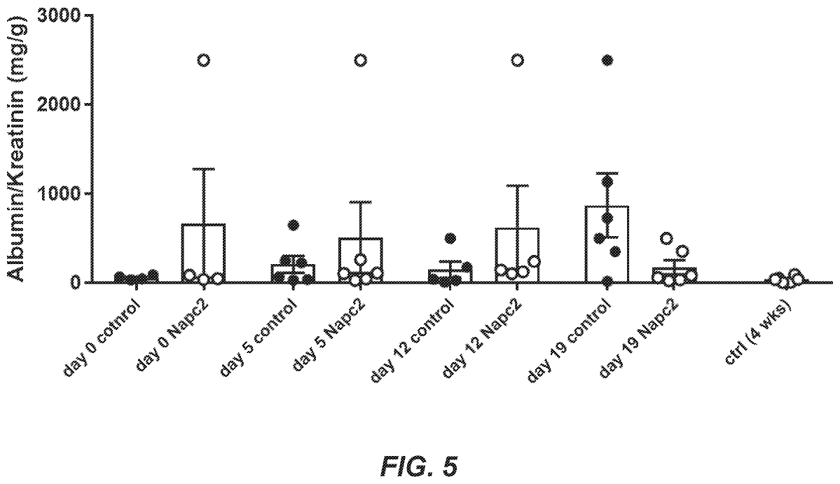

Renal function was assessed by measuring albumin in the urine during therapy with rNAPc2. Sham treated control mice developed progressive albuminuria during the observation period, whereas albumin levels in urine did not increase in rNAPc2 treated mice (FIG. 5). One mouse with high baseline albuminuria was randomized to the rNAPc2 treatment group but showed reduced albuminuria at the end of the experiment. These data indicated maintenance of kidney function in rNAPc2 treated MRL/lpr mice.

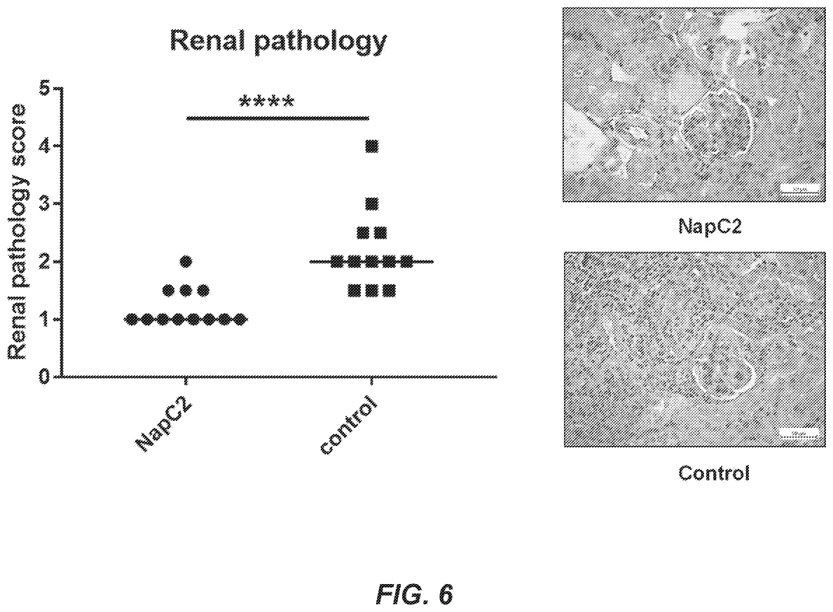

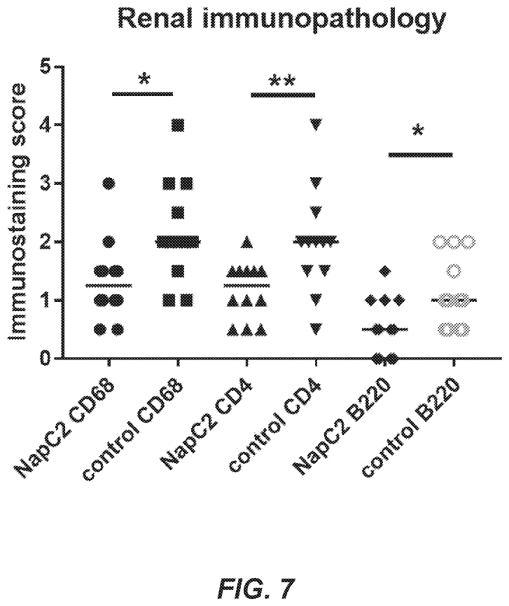

Kidney pathology was evaluated in the two treatment cohorts by histology. The renal pathology score, a composite of glomerular and tubular injury, was significantly improved in rNAPc2 versus control mice (FIG. 6). In addition, immune cell infiltration was semi-quantitively scored for macrophages (CD68), T cells (CD4), and B cells (B220) on sections processed for immuno-histochemistry. Treatment with rNAPc2 significantly reduced kidney immune cell infiltration (FIG. 7).

These data document an inhibitory effect of rNAPc2 on aPL signaling in monocytes and the prevention of autoimmune disease in relevant preclinical model of SLE. The suppression of B cell expansion responsible for the production of aPL reactive with both cardiolipin and β2GPI provides a rationale for therapeutic intervention in patients with severe antiphospholipid syndrome and the reduction of kidney pathology in a mouse model with SLE like pathology supports broader therapeutic actions of rNAPc2 in the prevention and reversal of end organ damage in autoimmune diseases.

### Example 3—NAPc2 is Superior to Anticoagulation with Heparin to Suppress the Development of aPL in Lupus-Prone Mice

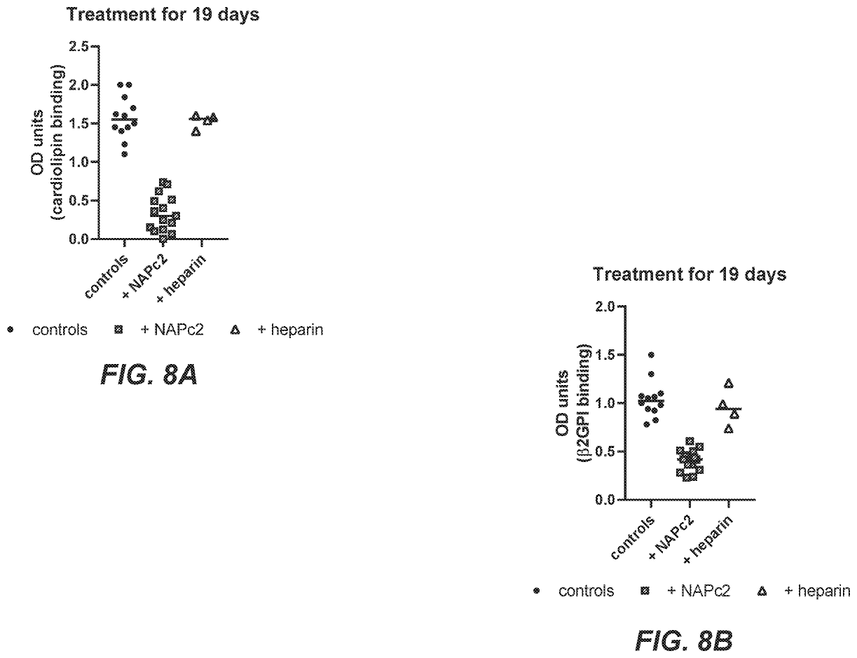

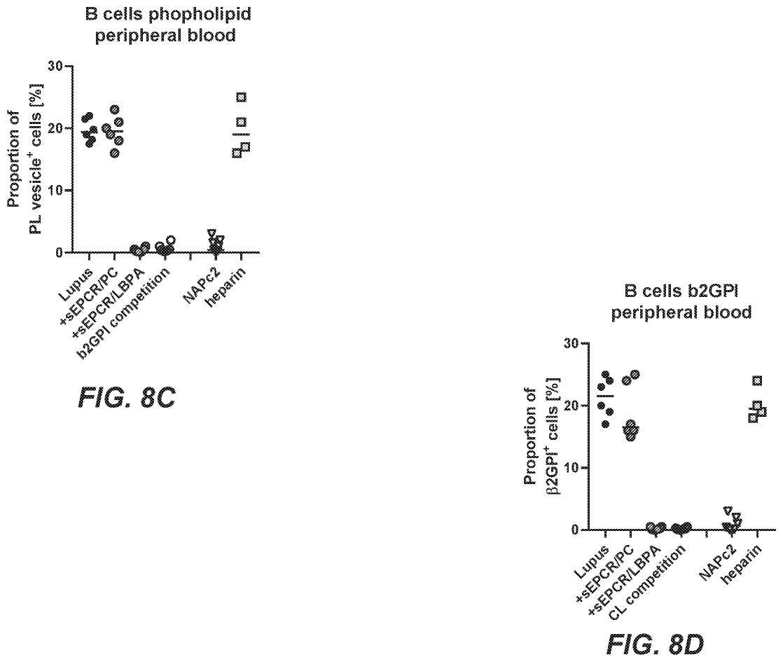

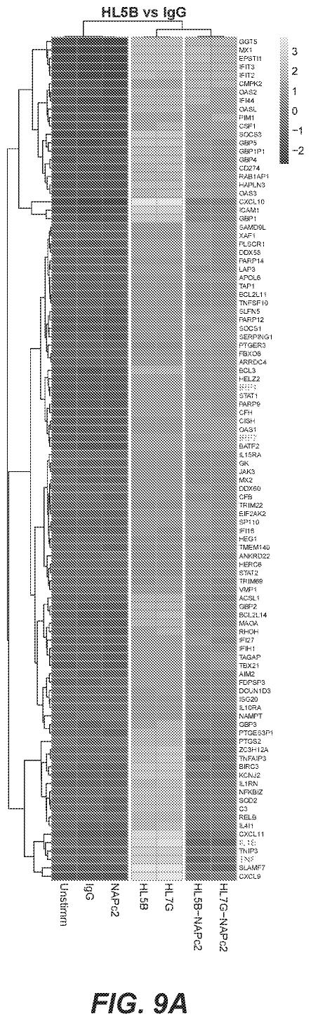

The therapeutic effects of anticoagulation with low molecular weight heparin (LMWH) versus NAPc2 treatment on the development of aPL and activation of circulating B1 cells reactive were compared with EPCR-LBPA. MRL/lpr mice were randomized at an age of 5 weeks to receive NAPc2 or a therapeutic dose of LMWH for 19 days. Whereas NAPc2 suppressed the development of antibodies reactive with cardiolipin as well as with β2GPI, heparin therapy was without effects on the antibody titers developing in MRL/lpr mice (FIGS. 8A and 8B). Peripheral blood was collected from these mice and was stained for phospholipid and β2GPI reactivity of B1 cells. NAPc2, but not heparin, suppressed the appearance of these B cells and staining was specifically prevented by EPCR-LBPA, but not unmodified EPCR (FIGS. 8C and 8D). These data showed that only NAPc2, but not standard heparin anticoagulation therapy, prevented the development of B cells recognizing EPCR-LBPA, the pathogenic target in lupus associated antiphospholipid syndrome.

### Example 4—NAPc2 Acts as a Modulator for aPL Signaling in Monocytes

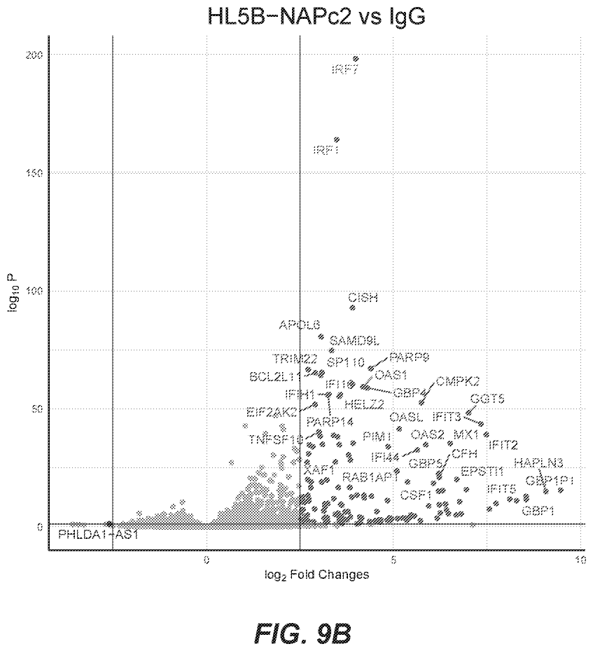

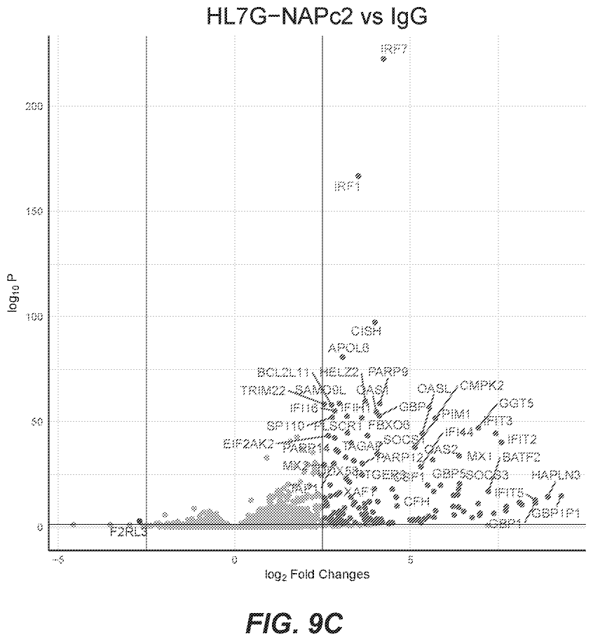

NAPc2 was shown to reprogram monocyte responses to aPL in a unique way. When monocytic MM1 cells were stimulated in plasma with human monoclonal antiphospholipid antibodies HL5B or HL7G, a similar transcriptional response was obtained (FIG. 9A). Inhibition of aPL signaling under these experimental conditions confirmed that NAPc2 effectively suppressed aPL proinflammatory responses and procoagulant TF upregulation (FIG. 9B). However, the induction of interferon signaling was not inhibited, as indicated by the upregulation of IRF1 and IRF7 (FIG. 9C). NAPc2 showed the same inhibitory profile when responses were induced with aPL HL7G that is cross-reactive with β2GPI and EPCR-LBPA (FIG. 2D, E). These data demonstrate that NAPc2 not simply acts as a suppressor of aPL signaling, but rather a response modifier with profound anti-inflammatory effects.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
1 Muller-Calleja N, Hollerbach A, Royce J, Ritter S, Pedrosa D, Madhusudhan T, Teifel S, Meineck M, Hauser F, Canisius A, Nguyen T S, Braun J, Bruns K, Etzold A, Zechner U, Strand S, Radsak M, Strand D, Gu J M, Weinmann-Menke J, Esmon C T, Teyton L, Lackner K J, Ruf W. Lipid presentation by the protein C receptor links coagulation with autoimmunity. Science. 2021; 371. 10.1126/science.abc0956.
2 Muller-Calleja N, Hollerbach A, Ritter S, Pedrosa D G, Strand D, Graf C, Reinhardt C, Strand S, Poncelet P, Griffin J H, Lackner K J, Ruf W. Tissue factor pathway inhibitor primes monocytes for antiphospholipid antibody-induced thrombosis. Blood. 2019; 134: 1119-31. 10.1182/blood.2019001530.
3 Muller-Calleja N, Ritter S, Hollerbach A, Falter T, Lackner K J, Ruf W. Complement C5 but not C3 is expendable for tissue factor activation by cofactor-independent antiphospholipid antibodies. Blood Adv. 2018; 2: 979-86. 10.1182/bloodadvances.2018017095.

What is claimed:

1. A method for treating a subject for an autoimmune or inflammatory condition, the method comprising administering to the subject a therapeutically effective amount of a pharmaceutical composition comprising nematode anticoagulant protein c2 (NAPc2) or NAPc2/proline.

2. The method of claim 1, wherein the pharmaceutical composition comprises NAPc2.

3. The method of claim 1, wherein the pharmaceutical composition comprises NAPc2/proline.

4. The method of any of claims 1-3, wherein the subject was determined to have symptoms of the autoimmune or inflammatory condition.

5. The method of claim 4, wherein the pharmaceutical composition is administered to the subject following the onset of symptoms.

6. The method of any of claims 1-3, wherein the subject does not have symptoms of the autoimmune or inflammatory condition.

7. The method of any of claims 1-6, wherein the pharmaceutical composition is administered prior to development of any symptoms of the autoimmune or inflammatory condition.

8. The method of any of claims 1-7, wherein the subject was previously treated for the autoimmune or inflammatory condition with a previous treatment.

9. The method of claim 8, wherein the subject was determined to be resistant to the previous treatment.

10. The method of any of claims 1-9, wherein the pharmaceutical composition is administered via subcutaneous injection.

11. The method of any of claims 1-9. wherein the pharmaceutical composition is administered via intravenous infusion.

12. The method of any of claims 1-11, wherein the pharmaceutical composition is administered to the subject every other day.

13. The method of any of claims 1-12, wherein the NAPc2 or NAPc2/proline is administered at a dose of between 5 μg/kg and 10 μg/kg.

14. The method of any of claims 1-13, wherein the NAPc2 or NAPc2/proline is administered at a dose of about 10 μg/kg.

15. The method of any of claims 1-13, wherein the NAPc2 or NAPc2/proline is administered at a dose of about 7.5 μg/kg.

16. The method of any of claims 1-13, wherein the NAPc2 or NAPc2/proline is administered at a dose of about 5 μg/kg.

17. The method of any of claims 1-13, wherein the method comprises administering the NAPc2 or NAPc2/proline at a dose of about 7.5 μg/kg on a first day, providing the NAPc2 or NAPc2/proline at a dose of about 5 μg/kg on a third day, and providing the NAPc2 or NAPc2/proline at a dose of about 5 μg/kg on a fifth day.

18. The method of any of claims 1-17, further comprising administering an additional anti-inflammatory agent to the subject.

19. The method of claim 18, wherein the additional anti-inflammatory agent is a non-steroidal anti-inflammatory drug.

20. The method of any of claims 1-19, further comprising administering an anticoagulant to the subject.

21. The method of claim 20, wherein the anticoagulant is a vitamin K epoxide reductase complex 1 (VKORC1) inhibitor, a thrombin inhibitor, or a factor Xa inhibitor.

22. The method of claim 20, wherein the anticoagulant is warfarin, heparin or synthetic analogs thereof, rivaroxaban, dabigatran, apixaban, or edoxaban.

23. The method of any of claims 1-19, wherein the method does not comprise administering an additional anticoagulant.

24. The method of any of claims 1-19, wherein the pharmaceutical composition does not comprise an additional anticoagulant.

25. The method of any of claims 1-24, wherein the autoimmune or inflammatory condition is systemic lupus erythematosus.

26. The method of any of claims 1-24, wherein the autoimmune or inflammatory condition is antiphospholipid syndrome.

27. The method of any of claims 1-26, wherein the subject was determined to have antiphospholipid antibodies.

28. The method of any of claims 1-26, further comprising, prior to administering to the subject the pharmaceutical composition, detecting the presence of antiphospholipid antibodies in the subject.

29. The method of claim 28, wherein detecting the antiphospholipid antibodies comprises an enzyme linked immunosorbent assay (ELISA).

30. A method for treating a subject for antiphospholipid syndrome, the method comprising administering to the subject a therapeutically effective amount of a pharmaceutical composition comprising nematode anticoagulant protein c2 (NAPc2) or NAPc2/proline.

31. The method of claim 30, wherein the pharmaceutical composition comprises NAPc2.

32. The method of claim 30, wherein the pharmaceutical composition comprises NAPc2/proline.

33. The method of any of claims 30-32, wherein the pharmaceutical composition is administered via subcutaneous injection.

34. The method of any of claims 30-32, wherein the pharmaceutical composition is administered via intravenous infusion.

35. The method of any of claims 30-34, wherein the pharmaceutical composition is administered to the subject every other day.

36. The method of any of claims 30-35, wherein the NAPc2 or NAPc2/proline is administered at a dose of between 5 μg/kg and 10 μg/kg.

37. The method of any of claims 30-36, wherein the NAPc2 or NAPc2/proline is administered at a dose of about 10 μg/kg.

38. The method of any of claims 30-36, wherein the NAPc2 or NAPc2/proline is administered at a dose of about 7.5 μg/kg.

39. The method of any of claims 30-36, wherein the NAPc2 or NAPc2/proline is administered at a dose of about 5 μg/kg.

40. The method of any of claims 30-36, wherein the method comprises administering the NAPc2 or NAPc2/proline at a dose of about 7.5 μg/kg on a first day, administering the NAPc2 or NAPc2/proline at a dose of about 5 μg/kg on a third day, and administering the NAPc2 or NAPc2/proline at a dose of about 5 μg/kg on a fifth day.

41. The method of claim 30-40, further comprising, prior to administering the pharmaceutical composition, detecting the presence of antiphospholipid antibodies in the subject.

42. The method of claim 41, wherein detecting the antiphospholipid antibodies comprises an enzyme linked immunosorbent assay (ELISA).

43. The method of any of claims 30-42, wherein the method does not comprise administering an additional anticoagulant.

44. The method of any of claims 30-42, wherein the pharmaceutical composition does not comprise an additional anticoagulant.

45. A method for treating a subject for systemic lupus erythematosus, the method comprising administering to the subject a therapeutically effective amount of a pharmaceutical composition comprising nematode anticoagulant protein c2 (NAPc2) or NAPc2/proline.

46. The method of claim 45, wherein the pharmaceutical composition comprises NAPc2.

47. The method of claim 45, wherein the pharmaceutical composition comprises NAPc2/proline.

48. The method of any of claims 45-47, wherein the pharmaceutical composition is administered via subcutaneous injection.

49. The method of any of claims 45-47, wherein the pharmaceutical composition is administered via intravenous infusion.

50. The method of any of claims 45-49, wherein the pharmaceutical composition is administered to the subject every other day.

51. The method of any of claims 45-50, wherein the NAPc2 or NAPc2/proline is administered at a dose of between 5 μg/kg and 10 μg/kg.

52. The method of any of claims 45-50, wherein the NAPc2 or NAPc2/proline is administered at a dose of about 10 μg/kg.

53. The method of any of claims 45-50, wherein the NAPc2 or NAPc2/proline is administered at a dose of about 7.5 μg/kg.

54. The method of any of claims 45-50, wherein the NAPc2 or NAPc2/proline is administered at a dose of about 5 μg/kg.

55. The method of any of claims 45-50, wherein the method comprises administering the NAPc2 or NAPc2/proline at a dose of about 7.5 μg/kg on a first day, administering the NAPc2 or NAPc2/proline at a dose of about 5 μg/kg on a third day, and administering the NAPc2 or NAPc2/proline at a dose of about 5 μg/kg on a fifth day.

56. The method of claim 45-55, further comprising, prior to administering the pharmaceutical composition, detecting the presence of antiphospholipid antibodies in the subject.

57. The method of claim 56, wherein detecting the antiphospholipid antibodies comprises an enzyme linked immunosorbent assay (ELISA).

58. The method of any of claims 45-57, wherein the method does not comprise administering an additional anticoagulant.

59. The method of any of claims 45-57, wherein the pharmaceutical composition does not comprise an additional anticoagulant.

60. A method for inhibiting antiphospholipid antibody-induced signaling in a cell comprising administering to the cell an effective amount of NAPc2.

61. A method for inhibiting antiphospholipid antibody-induced signaling in a cell comprising administering to the cell an effective amount of NAPc2/proline.


SEQUENCE LISTING (2 sequences)

SEQ ID NO: 1 - protein, 84 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRN

SEQ ID NO: 2 - protein, 85 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRNP